(19) United States
(12) Patent Application Publication
(10) Pub. No.: US 2015/0351687 A1
(43) Pub. Date: Dec. 10, 2015

(54) EMBEDDED ACTIVE ACTUATOR DRIVE SYSTEM

(71) Applicant: HITS CO., LTD., Geumcheon-gu, Seoul (KR)

(72) Inventors: Jong-Jeong KIM, Incheon (KR); Yong-Han CHUN, Incheon (KR); Sun-Yeol LEE, Seoul (KR); Hyun-Su HONG, Chungju-si (KR)

(21) Appl. No.: 14/760,136

(22) PCT Filed: Aug. 26, 2014
(86) PCT No.: PCT/KR2014/007913

§ 371 Date: Jul. 9, 2015

(30) Foreign Application Priority Data
Oct. 8, 2013 (KR) 10-2013-0119611

(51) Int. Cl. A61B 5/00; H04W 4/00; A61B 5/0205; H04B 1/3827

(52) U.S. Cl. CPC A61B 5/6802; H04B 1/385; H04W 4/008; A61B 5/0002; A61B 5/02055; A61B 5/024

(57) ABSTRACT

Provided is an embedded active actuator drive system. In an actuator drive system for controlling an actuator for operating an actuator material that is sensitised to a human body, provided is an embedded active actuator drive system that can actively control an actuator mounted in the particular smart sheet according to bio-signal information that is detected in real-time by linking with a database drive information about driving the actuator that is mounted in the particular smart sheet and that operates the actuator material sensitised to a human body.

### TECHNICAL FIELD

The present disclosure relates to an embedded active actuator drive system, and more particularly, to an embedded active actuator drive system that stores, in a DB unit, drive information on an actuator operating a drive material embedded in a particular smart sheet and reacting to a human body, senses biometrical signal information on the particular smart sheet in real time by using a textile-based array sensor embedded in the particular smart sheet, and compares the biometrical signal information on the particular smart sheet with the drive information on the actuator related to the particular smart sheet store din the DB unit to drive the actuator embedded in the particular smart sheet when the biometrical signal information on the particular smart sheet stored in the DB unit exceeds the threshold of the drive information stored in the DB unit.

### BACKGROUND ART

FIG. 1 is a schematic diagram of a typical actuator drive system having a biometrical signal sensor.

As shown in FIG. 1, the actuator drive system having the typical biometrical signal sensor includes a sensor unit 1 senses a biometrical signal, a control unit 2 receiving the biometrical signal sensed by the sensor unit 1 and comparing the biometrical signal with the threshold of drive information for driving an actuator stored in a control unit to determine whether to drive the actuator, and a drive unit 3 receiving a control signal for driving the actuator from the control unit 2 and driving the actuator.

In this example, the typical actuator drive system is automatically driven irrespective of a user when the same threshold assigned to the control unit 2 is exceeded. That is, since the actuator is driven based on the same value assigned to the control unit 2 even when the threshold of actuator drive information required varies according to a user, there was a limitation in that it is difficult to actively operate according to a user.

Also, since the sensor unit, the control unit and the drive unit are connected by a data cable, the typical actuator drive system has a limitation in that the performance of the system decreases or the system stops when the data cable experiences an external shock.

(Patent Literature 1) Korean Patent No. KR 10-0609077 (published on Jul. 27, 2006), Line 21 of Page 3 to Line 10 of Page 5, and FIG. 1.

### DISCLOSURE OF THE INVENTION

### Technical Problem

Embodiments provide an embedded active actuator drive system that may actively control an actuator embedded in a particular smart sheet according to biometrical signal information on the particular smart sheet sensed in real time by linking drive information driving an actuator operating a drive material embedded in the particular smart sheet and reacting to a human body to a DB.

### Technical Solution

In one embodiment, an actuator drive system for controlling an operation of an actuator driving a drive material responsive to a human body includes at least one sensor unit embedded in a particular smart sheet with which the actuator is combined, wherein the at least one sensor unit comprises a textile-based array sensor for sensing various kinds of biometrical signals, generates biometrical signal data on the particular smart sheet in real-time, and has unique identification information, a DB unit storing particular smart sheet information corresponding to the unique identification information on the sensor unit and drive information for driving the actuator in the particular smart sheet, a control unit using the biometrical signal data and the unique identification information received from the at least one sensor unit to generate biometrical signal information on the particular smart sheet, and comparing the biometrical signal information on the particular smart sheet with the drive information on the actuator for the particular smart sheet stored in the DB to generate a control signal for controlling the actuator, and a drive unit controlling the operation of the actuator in the smart sheet according to the control signal received from the control unit and stopping the operation of the actuator when an emergency occurs, wherein the sensor unit, the DB unit, the control unit and the drive unit mutually transmit and receive signals by using Bluetooth wireless communication consuming less power.

### Advantageous Effects

As described above, the inventive concept provides an embedded active actuator drive system that may actively control an actuator embedded in a particular smart sheet according to biometrical signal information on the particular smart sheet sensed in real time by linking drive information driving an actuator operating a drive material embedded in the particular smart sheet and reacting to a human body to a DB.

### BRIEF DESCRIPTION OF THE DRAWINGS

FIG. 1 is a schematic diagram of a typical actuator drive system having a biometrical signal sensor.

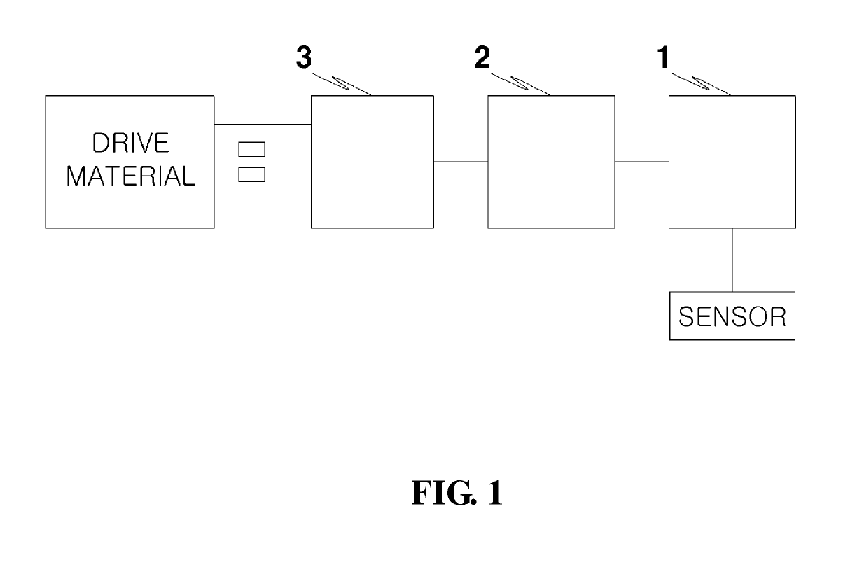

FIG. 2 is a block diagram of an embedded active actuator drive system according to an embodiment of the inventive concept.

FIG. 3 is a block diagram of a sensor unit in FIG. 2.

FIG. 4 is a block diagram of a control unit in FIG. 2.

FIG. 5 is a block diagram of a drive unit in FIG. 2.

FIG. 6 is a flowchart of an operation process of an active actuator drive system according to an embodiment of the inventive concept.

### MODE FOR CARRYING OUT THE INVENTION

The inventive concept relates to an actuator drive system that drives a drive material reacting to a human body and controls the operation of an actuator combined to a smart sheet.

The material of the smart sheet herein is a concept similar to an intelligent material having a sensor function, means a material capable of reacting according to a change in external environment and has a characteristic that it is possible to satisfy various needs according to a recent sharp change in industrial environment.

To additionally describe the material of the smart sheet, functional fiber may be inserted into each of multiple layers that are formed in the structure of a plurality of layers.

In this case, the functional fiber includes a high-strength conductive thread, a fiber-based sensor thread, optical fiber, a ceramic thread, a carbon thread and a metal thread and may be fabricated in the structure of a plurality of layers.

Since the functional fiber is disposed in a net shape, it is possible to make one functional fiber have a wide area, prevent the rupture of the functional fiber, increase durability and manufacture a smart sheet more simply and cheaply by using a typical fabric fabrication technology and a direct printing process that performs printing with various functional inks having electrical characteristics on a polymer substrate or fabric.

The smart sheet utilizing such a smart material may be roughly divided into a passive type and an active type, and the active type may be applied to an embodiment of the inventive concept.

The active smart sheet may include a sensor capable of sensing a change in environment and an actuator capable of reacting to the sensor.

Also, the sensor applied to the active smart sheet has a structure in which it is embedded or fixed into a thin polymer sheet, paper, fiber material, or a special material in the form of an array chip in order to sense the physiological change of a body by using an electrical, magnetic or optical signal, in which case a biometrical signal responsive actuator operating according to a sensed biometrical signal is embedded into the same sheet (material). Also, it is also possible to drive an actuator installed in a separate structure, by using the sensed biometrical signal in communication with an external digital device, depending on the situation.

In this example, the active smart sheet has a fundamental body protection function such as impact resistance, heat resistance or cold resistance and may drive the actuator having a physical function to spontaneously cope with an environment change inside or outside a body, when it is determined that a biometrical signal measured in real time is dangerous for a human body.

The smart sheet technology according to the inventive concept to which the biometrical signal sensing and active actuator drive system is applied is obtained by combining a textile based sensor with the core technology of an NT, IT and BT, is thus a hybrid technology having sensing and actuating technologies, and may be applied to various fields such as healthcare, safety, security and robot technologies.

Embodiments according to the inventive concept are described below in detail with reference to the accompanying drawings.

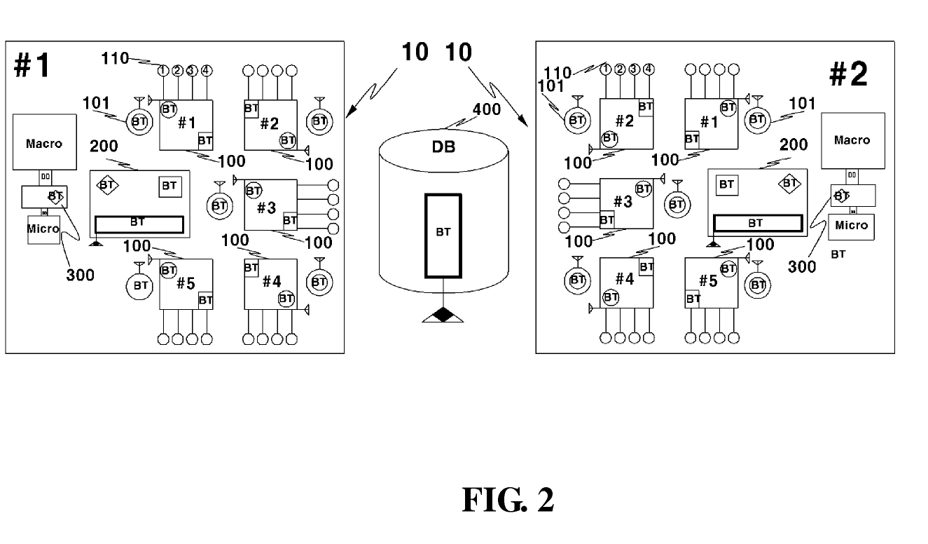

FIG. 2 is a block diagram of an embedded active actuator drive system according to an embodiment of the inventive concept.

As shown in FIG. 2, the embedded active actuator drive system according to the embodiment of the inventive concept includes a sensor unit 100, a control unit 200, a drive unit 300, and a DB unit 400.

In the embodiment of the inventive concept, the sensor unit 100, the control unit 200, and the drive unit 300 are implemented in an embedded fashion, i.e., integrally embedded into a particular smart sheet 10 and the DB unit 400 includes a DB server that links information on the actuator drive system and the particular smart sheet into which the actuator drive system is embedded.

More particularly, the sensor unit 100 is embedded into the particular smart sheet with which an actuator is combined, includes a textile-based array sensor 110 in order to sense various kinds of biometrical signals, generates biometrical signal data on the particular smart sheet in real time and has unique identification information. In the embodiment of the inventive concept, the sensor unit 100 is formed in plurality in the particular smart sheet as shown in FIG. 2.

Also, the DB unit 400 stores particular smart sheet information corresponding to the unique identification information on the sensor unit 100, biometrical signal information on the particular smart sheet, and drive information for driving the actuator in the particular smart sheet.

In addition, the control unit 200 uses the biometrical signal data and the unique identification information received from one or more sensor units 100 to generate the biometrical signal information on the particular smart sheet, and compares the biometrical signal information on the particular smart sheet with the drive information on the actuator for the particular smart sheet stored in the DB unit 400 to generate a control signal controlling the actuator.

Also, the drive unit 300 controls the operation of the actuator in the smart sheet 10 according to the control signal received from the control unit 200 and stops the operation of the actuator when an emergency occurs.

The sensor unit 100, the DB unit 400, the control unit 200, and the drive unit 300 of the actuating system according to the embodiment of the inventive concept mutually receive and transmit signals by using Bluetooth wireless communication which consumes less power.

The Bluetooth communication which is applied to the inventive concept uses 2400 MHz to 2483.5 MHz that is an industrial scientific and medical (ISM) frequency band. In this example, the ISM is a frequency band allocated for industry, science and medicine and is commonly used for a personal wireless device that emits low-power radio wave because there is no need to receive a permission to use a radio wave.

Since the Bluetooth communication uses a frequency hopping method, it needs synchronization between devices for communication and thus has a master-slave configuration, and in the embodiment of the inventive concept, the master-slave configuration may be applied to between the sensor unit 100 and an external sensor 101, between the control unit 200 and the sensor unit 100, between the control unit 200 and the drive unit 300, and between the DB unit 400 and the control unit 200 so that pairing (which means binding two devices to work together as a pair) is enabled when power is supplied.

Also, when the Bluetooth technology which is applied to the embodiment of the inventive concept is applied to between the sensor unit 100 and the external sensor 101, between the control unit 200 and the sensor unit 100, and between the control unit 200 and the drive unit 300, Bluetooth 4.0 technology which maintains a data rate of 24 Mbps and requires significantly decreased power is applied, and when the Bluetooth technology is applied to between the DB unit 400 and the control unit 200, Bluetooth 2.0 technology may be used due to the DB unit 400 being a DB server that requires higher power consumption than the Bluetooth 4.0 technology.

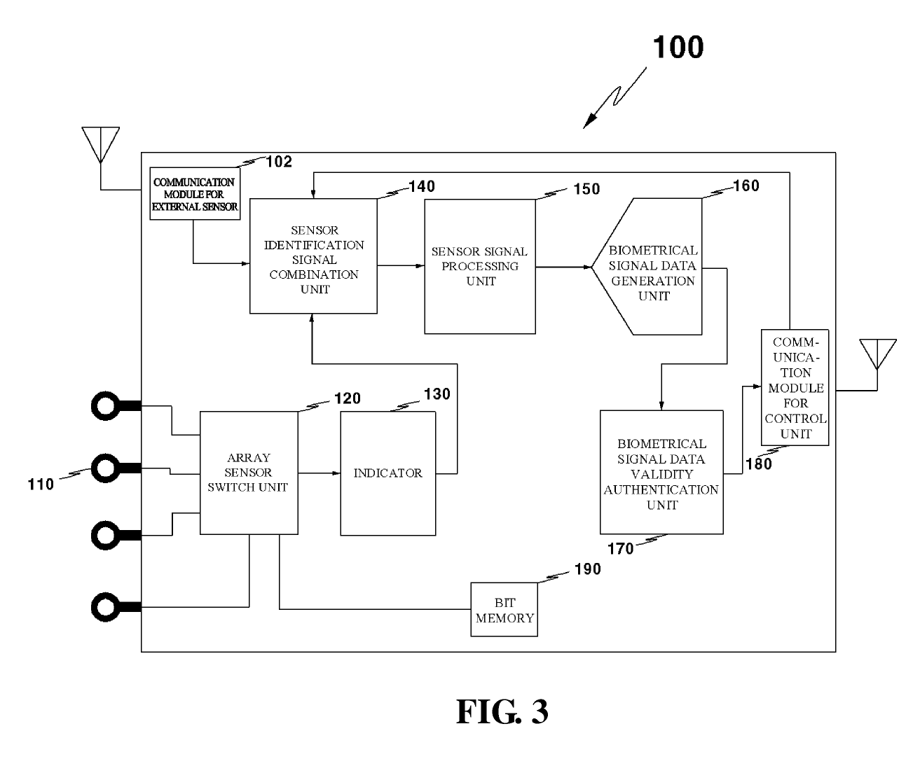

FIG. 3 is a block diagram of a sensor unit in FIG. 2.

The sensor unit according to the embodiment of the inventive concept is described below with reference to FIG. 3.

As shown in FIG. 3, the sensor unit 100 configuring the embedded active actuator drive system according to the embodiment of the inventive concept includes an array sensor 110, an array sensor signal switch unit 120, an indicator 130, a sensor identification signal combination unit 140, a sensor signal processing unit 150, a biometrical signal a data generation unit 160, a biometrical signal data validity authentication unit 170, a communication module 180 for a control unit, a communication module 102 for external sensor, and a bit memory.

More particularly, the array sensor 110 is a textile-based sensor that is formed in such a manner that a plurality of fiber-based environment responsive sensors are arranged in a net shape in order to sensor various kinds of biometrical signals including size of range that a single sensor may not measure.

Also, the array sensor signal switch unit 120 may simultaneously receive, from the array sensor 110, various kinds of biometrical signals such as body temperature, pressure, a heart rate, blood pressure, respiration and humidity and in order to decrease power consumption, it selects only a required sensor from among a plurality of sensors configuring the array sensor 110 to generate an array sensor signal.

In this case, the bit memory 190 may play a role in storing, to a 2-bit unit, the number of arrays of the sensors selected by the array sensor signal switch unit 120 from among the plurality of sensors configuring the array sensor 110, and regularly resetting.

Also, the indicator 130 may amplify the size of an array sensor signal generated by the array sensor signal switch unit 120 or adjust the size of amplitude to identify the array sensor signal according to the kind of the biometrical signal.

In addition, the sensor identification signal combination unit 140 combines the unique sensor identification signal of the sensor unit 100 capable of being identified by the control unit 200 with the array sensor signal identified by various kinds of biometrical signals passing through the indicator 130 to generate an analog sensor signal unique to the sensor unit 100. In this example, the sensor identification number combination unit 140 combines an MAC address stored in a communication module using Bluetooth wireless communication in the sensor unit 100 with a unique identification signal to generate an analog sensor signal unique to the sensor unit 100, and may use the MAC addresses of the communication module 102 for an external sensor and the communication module 180 for the control unit as shown in FIG. 3.

Also, the sensor signal processing unit 150 amplifies an analog signal passing through the sensor identification signal combination unit 140 and removes noise, the biometrical signal data generation unit 160 converts the analog sensor signal passing through the sensor signal processing unit 150 into a digital data signal to generate biometrical signal data and the biometrical signal data validity authentication unit 170 checks the format and error of the biometrical signal data generated by the biometrical signal data generation unit 160 to authenticate data validity.

The communication module 180 for the control unit uses Bluetooth wireless communication to play a role in transmitting, to the control unit 210, the biometrical signal data passing through the biometrical signal data validity authentication unit 170.

Also, the communication module 102 for the external sensor in the sensor unit 100 according to an embodiment of the inventive concept uses Bluetooth wireless communication consuming less power in order to input an external sensor signal generated by the external sensor 101 to the sensor identification number combination unit 140, in which case the external sensor signal may be a biometrical signal of a different kind from the biometrical signal sensed by the array sensor 110.

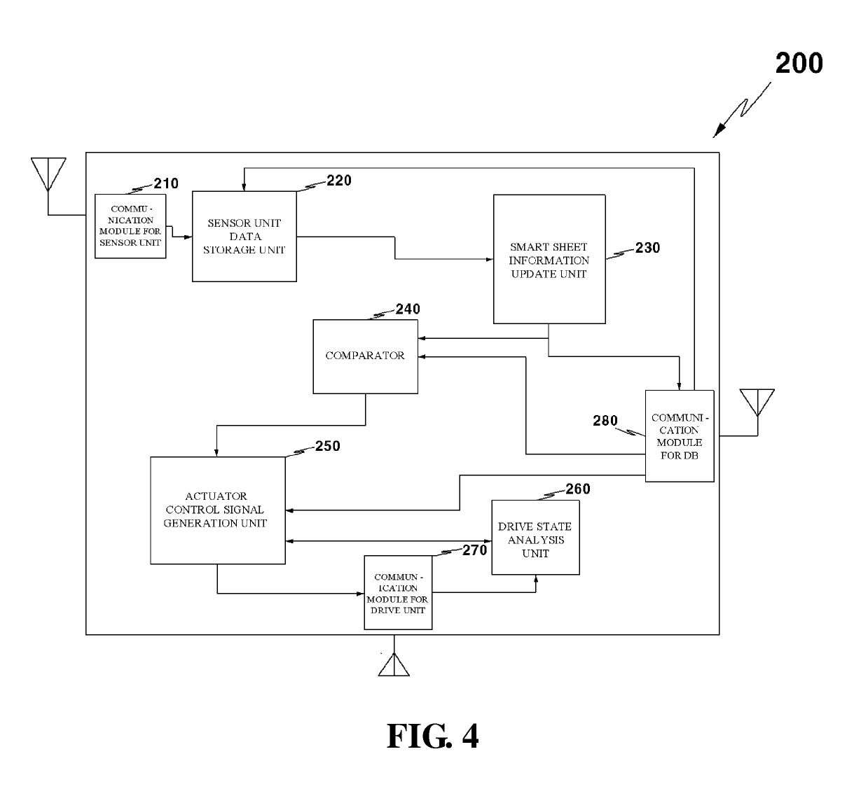

FIG. 4 is a block diagram of a control unit in FIG. 2.

The control unit 200 according to the embodiment of the inventive concept is described below with reference to FIG. 4.

As shown in FIG. 4, the control unit 200 configuring the embedded active actuator drive system according to the embodiment of the inventive concept includes a communication module 210 for a sensor unit, a sensor unit data storage unit 220, a smart sheet information update unit 230, a comparator 240, an actuator control signal generation unit 250, a communication module 280 for a DB, a communication module 270 for a drive unit, and a drive state analysis unit 260.

More particularly, the communication module 210 for the sensor unit receives, in real time from the sensor unit 100, biometrical signal data with which unique identification information identifying a particular sensor unit is combined, and the sensor unit data storage unit 220 combines unique identification information combined with the biometrical signal data of the particular sensor unit in real time received from the communication module 210 for the sensor unit and particular smart sheet information corresponding to the unique identification information on the sensor unit 100 stored in the DB unit 400 to generate the biometrical signal data received from the sensor unit 100 as biometrical signal information on a particular smart sheet and store it.

Also, the smart sheet information update unit 230 stores the biometrical signal information on the particular smart sheet 10 stored in the sensor unit data storage unit 220 in the DB unit 400 for an update.

In the embodiment of the inventive concept, the comparator 240 compares the biometrical signal information on the particular smart sheet with drive information on an actuator corresponding to the particular smart sheet received from the DB unit 400 to determine whether the biometrical signal information on the particular smart sheet is beyond the threshold of the drive information stored in the DB unit 400, and when the comparator 240 determines that the biometrical signal information on the particular smart sheet is beyond the threshold of the drive information stored in the DB unit 400, the actuator control signal generation unit 250 receives, from the DB unit 400, drive information for driving the actuator, selects the type of the actuator in the drive information and generates a control signal for driving the selected actuator.

Also, the communication module 280 for the DB plays roles in transmitting, to the DB unit 400, the biometrical signal information on the particular smart sheet received from the smart sheet information update unit 230, and receiving, from the DB unit 400, drive information on an actuator corresponding to the particular smart sheet required by the comparator 240 and the actuator control signal generation unit 250, and particular smart sheet information corresponding to unique identification information on the sensor unit 100 required by the sensor unit data storage unit 220.

In addition, the communication module 270 for the drive unit transmits, to the drive unit 300, the control signal generated by the actuator control signal generation unit 250 and receives, from the drive unit 300, drive state information related to the current operation state of an actuator, and the drive state analysis unit 260 compares actuator drive information from the actuator control signal generation unit 250 with drive state information on an actuator received through the communication module 270 for the drive unit, generates a control signal stopping the operation of the actuator when they are different from each other, and transmits the control signal stopping the operation of the actuator to the drive unit 300 through the communication module 270 for the drive unit.

Also, the embodiment of the inventive concept is characterized in that the communication module 210 for the sensor unit, the communication module 280 for the DB, and the communication module 270 for the drive unit use Bluetooth wireless communication consuming less power as described above.

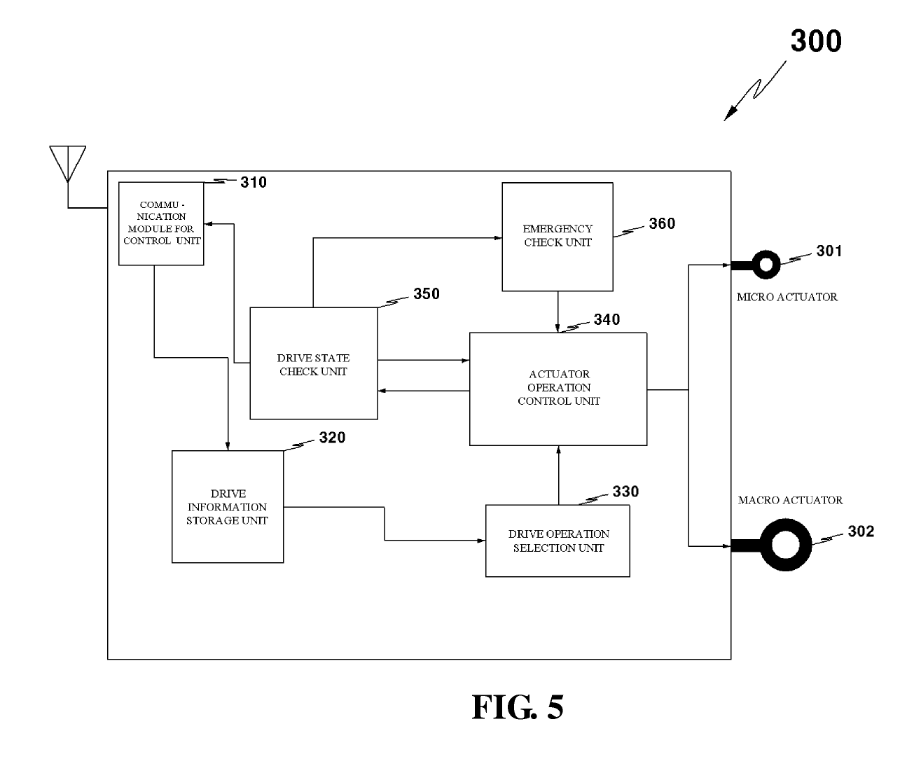

FIG. 5 is a block diagram of a drive unit in FIG. 2.

As shown in FIG. 5, the drive unit 200 configuring the embedded active actuator drive system according to the embodiment of the inventive concept includes actuators 301 and 302, an actuator operation control unit 340, a drive state check unit 350, a communication module 310 for a control unit, a drive information storage unit 320, a drive operation selection unit 330, and an emergency check unit 360.

The drive unit according to the embodiment of the inventive concept is described below in more detail with reference to FIG. 5.

The actuators 301 and 302 are embedded into a particular smart sheet in order to activate a drive material responsive to a human body and operate as any one of a micro actuator 301 and a macro actuator 302 by the control of the actuator operation control unit 340. In this case, the micro actuator 301 typically requires a relatively weak force in comparison to the macro actuator 302.

Also, the drive state check unit 350 receives, from the actuator operation control unit 340, drive state information representing the presence or absence of the operation of the actuator 301 or 302 in order to perform a feedback check, and the communication module 310 for the control unit uses Bluetooth wireless communication consuming less power to transmit, to the control unit 200, drive state information on the actuator 301 or 302 received from the drive state check unit 350 and receive, from the control unit 200, a control signal for controlling the operation of the actuator 301 or 302.

When the communication module 310 for the control unit receives, from the control unit 200, the control signal for driving the selected actuator 301 or 302, the drive information storage unit 320 stores specific drive information including the type of the actuator included in the control signal, and the drive operation selection unit 330 receives the type of the actuator stored in the drive information storage unit 320, controls the actuator operation control unit 340 and selects any one of the micro actuator and the macro actuator.

Also, the emergency check unit 360 plays roles in transmitting control signals for stop and reset operations to the actuator operation control unit 340 and stopping the operation of the actuator 301 or 302 or resetting, when it receives a check-disable state signal from the drive state check unit 350.

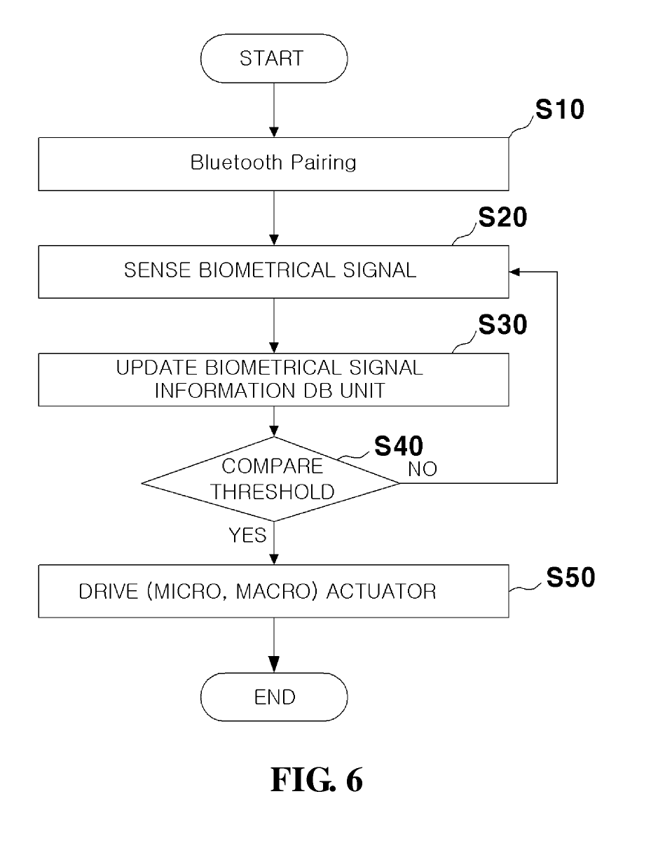

FIG. 6 is a flowchart of an operation process of an active actuator drive system according to an embodiment of the inventive concept.

The operation process of the active actuator drive system according to the embodiment of the inventive concept is described below with reference to FIG. 6.

As shown in FIG. 6, according to the active actuator drive system according to the embodiment of the inventive concept, the Bluetooth communication modules of the sensor unit 100, the control unit 200, the drive unit 300, and the DB unit 400 configuring the system are first paired in step S10 when power is supplied.

Next, the textile-based array sensor 110 in the sensor unit 100 generates biometrical signal data on a particular smart sheet in real time and transmits the data to the control unit 200 in step S20.

Also, the control unit 200 uses the biometrical signal data received from the sensor unit 100 to generate biometrical signal information on the particular smart sheet and update the DB unit 400 in step S30.

Then, the control unit 200 compares the biometrical signal information on the particular smart sheet with drive information on an actuator for the particular smart sheet stored in the DB unit 400 to determine whether to be beyond a threshold for driving an actuator in step S40.

Next, the drive unit 300 receives a control signal from the control unit 200 and operates a micro actuator or macro actuator.

As described above, the inventive concept provides an embedded active actuator drive system that may actively control an actuator embedded in a particular smart sheet according to biometrical signal information on the particular smart sheet sensed in real time by linking drive information driving an actuator operating a drive material embedded in the particular smart sheet and reacting to a human body to a DB.

Although the inventive concept has been described in detail so far, the applicant makes clear that the embodiments mentioned in the process of description are only exemplary and are not limitative and the variations of the components of the inventive concept that may be equivalently replaced would belong to the scope of the inventive concept when they do not depart from the technical spirit or field of the inventive concept provided by the following claims.

### INDUSTRIAL APPLICABILITY

The inventive relates to an embedded active actuator drive system field that may actively control an actuator embedded in a particular smart sheet according to biometrical signal information on the particular smart sheet sensed in real time by linking drive information driving an actuator operating a drive material embedded in the particular smart sheet and reacting to a human body to a DB, and according to the inventive concept, a smart sheet technology to which the biometrical signal sensing and active actuator drive system is applied is obtained by combining a textile based sensor with the core technology of an NT, IT and BT, is thus a hybrid technology having sensing and actuating technologies, and may be applied to various fields such as healthcare, safety, security and robot technologies.

1. An actuator drive system for controlling an operation of an actuator driving a drive material responsive to a human body, the actuator drive system comprising:
at least one sensor unit embedded in a particular smart sheet with which the actuator is combined, wherein the at least one sensor unit comprises a textile-based array sensor for sensing various kinds of biometrical signals, generates biometrical signal data on the particular smart sheet in real-time, and has unique identification information;
a DB unit storing particular smart sheet information corresponding to the unique identification information on the sensor unit and drive information for driving the actuator in the particular smart sheet;
a control unit using the biometrical signal data and the unique identification information received from the at least one sensor unit to generate biometrical signal information on the particular smart sheet, and comparing the biometrical signal information on the particular smart sheet with the drive information on the actuator for the particular smart sheet stored in the DB to generate a control signal for controlling the actuator; and
a drive unit controlling the operation of the actuator in the smart sheet according to the control signal received from the control unit and stopping the operation of the actuator when an emergency occurs,
wherein the sensor unit, the DB unit, the control unit and the drive unit mutually transmit and receive signals by using Bluetooth wireless communication consuming less power.

2. The embedded active actuator drive system of claim 1, wherein the sensor unit comprises:
a textile-based array sensor formed in a manner that a plurality of fiber-based environment responsive sensors are arranged in a net shape to sensor various kinds of biometrical signals including a size of range that it is difficult for a single sensor to measure;
an array sensor signal switch unit simultaneously receiving, from the array sensor, various kinds of biometrical signals comprising body temperature, pressure, a heart rate, blood pressure, respiration and humidity, wherein the array sensor signal switch unit selects only a required sensor from among a plurality of sensors configuring the array sensor to decrease power consumption and generates an array sensor signal;
an indicator amplifying a size of an array sensor signal generated by the array sensor signal switch unit or adjusting a size of amplitude to identify the array sensor signal according to the kind of the biometrical signal;
a sensor identification signal combination unit combining a sensor identification signal unique to the sensor unit capable of being identified by the control unit with the array sensor signal identified by various kinds of biometrical signals passing through the indicator to generate an analog sensor signal unique to the sensor unit;
a sensor signal processing unit amplifying the analog signal passing through the sensor identification signal combination unit and removing noise;
a biometrical signal data generation unit 160 converting the analog sensor signal passing through the sensor signal processing unit into a digital data signal to generate biometrical signal data;
a biometrical signal data validity authentication unit checking a format and error of the biometrical signal data generated by the biometrical signal data generation unit to authenticate data validity; and
a communication module for the control unit using Bluetooth wireless communication to transmit, to the control unit, the biometrical signal data passing through the biometrical signal data validity authentication unit.

3. The embedded active actuator drive system of claim 2, further comprising a communication module for an external sensor using Bluetooth wireless communication consuming less power to receive an external sensor signal generated by the external sensor to input the external sensor signal to the sensor identification number combination unit.

4. The embedded active actuator drive system of claim 3, wherein the external sensor signal is a biometrical signal of a different kind from a biometrical signal sensed by the array sensor.

5. The embedded active actuator drive system of claim 2, wherein the sensor identification number combination unit combines an MAC address stored in a communication module using Bluetooth wireless communication in the sensor unit with a unique identification signal to generate an analog sensor signal unique to the sensor unit.

6. The embedded active actuator drive system of claim 2, wherein the sensor unit further comprises a bit memory storing, to a 2-bit unit, the number of arrays of the sensors selected by the array sensor signal switch unit from among the plurality of sensors configuring the array sensor.

7. The embedded active actuator drive system of claim 1, wherein the control unit comprises:
a communication module for the sensor unit receiving, in real time from the sensor unit, biometrical signal data with which unique identification information identifying a particular sensor unit is combined;
a sensor unit data storage unit combining unique identification information combined with the biometrical signal data of the particular sensor unit in real time received from the communication module for the sensor unit and particular smart sheet information corresponding to the unique identification information on the sensor unit stored in the DB unit to generate the biometrical signal data received from the sensor unit as biometrical signal information on a particular smart sheet and store the generated information;
a smart sheet information update unit storing, the biometrical signal information on the particular smart sheet stored in the sensor unit data storage unit, in the DB unit 400 for an update;
a comparator comparing the biometrical signal information on the particular smart sheet with drive information on an actuator corresponding to the particular smart sheet received from the DB unit 400 to determine whether the biometrical signal information on the particular smart sheet is beyond a threshold of the drive information stored in the DB unit;
an actuator control signal generation unit receiving, from the DB unit, drive information for driving the actuator, selecting a type of an actuator included in the drive information and generating a control signal for driving a selected actuator, when the comparator determines that the biometrical signal information on the particular smart sheet is beyond the threshold of the drive information stored in the DB unit;
a communication module for a DB transmitting, to the DB unit, the biometrical signal information on the particular smart sheet received from the smart sheet information update unit, and receiving, from the DB unit, drive information on an actuator corresponding to the particular smart sheet required by the comparator and the actuator control signal generation unit, and particular smart sheet information corresponding to unique identification information on the sensor unit required by the sensor unit data storage unit;
a communication module for the drive unit transmitting, to the drive unit, the control signal generated by the actuator control signal generation unit and receiving, from the drive unit, drive state information related to a current operation state of an actuator; and
a drive state analysis unit comparing actuator drive information from the actuator control signal generation unit with drive state information on an actuator received through the communication module for the drive unit, generating a control signal stopping an operation of the actuator when they are different from each other, and transmitting the control signal stopping the operation of the actuator to the drive unit through the communication module for the drive unit.

8. The embedded active actuator drive system of claim 7, wherein each of the communication module for the sensor unit, the communication module for the DB and the communication module for the drive unit uses Bluetooth wireless communication consuming less power.

9. The embedded active actuator drive system of claim 1, wherein the drive unit comprises:
an actuator embedded in a particular smart sheet to activate a drive material responsive to a human body and operating as any one of a micro actuator and a macro actuator;
an actuator operation control unit controlling an operation of the actuator;
a drive state check unit receiving, from the actuator operation control unit, drive state information representing presence or absence of the operation of the actuator to perform a feedback check;
a communication module for the control unit using Bluetooth wireless communication consuming less power to transmit, to the control unit, drive state information on the actuator received from the drive state check unit and receive, from the control unit, a control signal for controlling the operation of the actuator;
a drive information storage unit storing specific drive information including a type of an actuator included in the control signal when the communication module for the control unit receives from the control unit the control signal for driving a selected actuator;
a drive operation selection unit receiving the type of the actuator stored in the drive information storage unit and enabling the actuator operation control unit to select any one of the micro actuator and the macro actuator; and
an emergency check unit transmitting control signals for stop and reset operations to the actuator operation control unit when receiving a check-disable state signal from the drive state check unit.
